(19) United States
(12) Patent Application Publication
(10) Pub. No.: US 2005/0063884 A1
(43) Pub. Date: Mar. 24, 2005

(54) Autoclave base including ventilated drawer

(76) Inventors: Jerry Sullivan, Cuyahoga Falls, OH (US); Walter E. Jahik, Willoughby, OH (US); Yelena Avrushchenko, Aurora, OH (US)

Correspondence Address: KATTEN MUCHIN ZAVIS ROSENMAN, 575 MADISON AVENUE, NEW YORK, NY, 10022-2585, US

(21) Appl. No.: 10/912,523

(22) Filed: Aug. 5, 2004

Related U.S. Application Data
(63) Continuation-in-part of application No. 10/464,011, filed on Jun. 18, 2003.
Application No. 10/464,011 is a continuation of application No. 10/183,238, filed on Jun. 27, 2002, now abandoned.
(60) Provisional application No. 60/493,843, filed on Aug. 8, 2003.
(60) Provisional application No. 60/554,190, filed on Mar. 18, 2004.

(51) Int. Cl. A61L 2/26

(52) U.S. Cl. 422/300; 422/307

(57) ABSTRACT

A support base for a front-loading sterilizer includes a retractable shelf for staging items to be loaded into the sterilizer, and for drying and cooling items that are removed from the sterilizer. The support base further includes a fan unit for directing an air flow from an interior cavity of the support base through a rear member of the support base to assist in drying and cooling the staged items. The fan unit is operated by a switch, which may positioned to be engaged by the shelf when the shelf is in a retracted position. The support base optionally includes a heating element for directing heat to the interior of the cavity. The support base may be constructed as an integral component of the sterilizer, integrally including at least one sterilizer chassis panel attached at a top edge of the support base.

### CROSS-REFERENCE TO RELATED APPLICATIONS

The present application is a continuation-in-part of and claims priority under 35 U.S.C. § 120 from U.S. patent application Ser. No. 10/464,011, which was filed on Jun. 18, 2003, and is a continuation application of U.S. patent application Ser. No. 10/183,238, filed on Jun. 27, 2002. The present application also claims priority under 35 U.S.C. § 119(e) from U.S. Provisional Patent Application Ser. No. 60/493,843, which was filed on Aug. 8, 2003, and from U.S. Provisional Patent Application Ser. No. 60/554,190, which was filed on Mar. 18, 2004. The disclosures of U.S. patent application Ser. Nos. 10/464,011 and 10/183,238 and U.S. Provisional Patent Application Nos. 60/493,843 and 60/554,190 are hereby incorporated by reference.

### FIELD OF THE INVENTION

This invention relates to a support base and work surface used in conjunction with industrial apparatus. More particularly, the invention relates to a sterilizer support base having a ventilated, retractable shelf.

### BACKGROUND OF THE INVENTION

Steam autoclaving is a well-known and reliable method used widely to sterilize media and lab equipment as well as to decontaminate infectious waste. Autoclaves introduce saturated steam within a pressurized chamber (for example, at pressures of 15 psi or more) in order to generate sufficient temperatures (for example, at or above 275 degrees Fahrenheit) to achieve sterilization. Heat produced by saturated steam at such temperatures is effective in damaging essential cellular structures of biological organisms present in the pressurized chamber, and with sufficient time, in killing such organisms.

Items placed in autoclave chambers are subjected to pressurized steam. Care is required in removing these items after the autoclaving process has completed. The chamber must be depressurized before opening. Upon removal, water condensate may be present on the sterilized items and require evaporation and/or draining. Also, the sterilized items may be slippery and difficult to handle.

Devices for holding items placed within an autoclave are disclosed in the prior art (for example, see U.S. Pat. No. 4,670,227, issued to Smith on Jun. 2, 1987), and specialized work surfaces for use externally with other appliances have also been disclosed (for example, see U.S. Pat. No. 4,436,356, issued to Stelling on Mar. 13, 1984). Disclosed in U.S. patent application Ser. No. 10/464,011 is specialized work surface positioned adjacent to an autoclave for receiving and drying sterilized items as they are removed from the autoclave chamber is.

In U.S. patent application S/N 10/464,011, an autoclave support base is disclosed that comprises a base housing having a top surface for receiving and supporting the autoclave, and left and right support members respectively and downwardly attached in proximity to left and right opposing edges of the top surface, such that the top surface and left and right support members define a cavity in the base housing. A holding shelf is slidably mounted within the cavity in the base housing.

The holding shelf includes a holding surface depressedly positioned with respect to front, rear, left and right edges of the holding shelf, so that surfaces defined between the front, rear, left and right edges of the holding shelf and the holding surface act to confine items placed on the holding surface. The holding surface may be pitched toward one or more perforations in the holding surface to facilitate drainage of condensate from sterilized items placed on the holding surface.

Items removed from the autoclave chamber and placed on the holding surface of the holding shelf may often retain significant amounts of heat and/or condensation. It would also be advantageous if the holding shelf incorporated an active ventilation system in order to increase the rate of drying of sterilized items placed on the holding surface and to provide for faster cooling of the sterilized items.

### SUMMARY OF THE INVENTION

A convenient work surface both for staging items to be placed in an autoclave for sterilizing, and for retaining items removed from the autoclave for staging, cooling and drying, is provided by a novel autoclave support base. The support base is equally well suited for application to other types of sterilizers such as dry-heat and chemical vapor sterilizers.

The support base comprises a base housing having a top member for receiving and supporting the autoclave, and left, right and rear members respectively and downwardly attached from the top member, such that the top member and the left, right and rear support members define a cavity in the base housing. A holding shelf is slidably mounted within the cavity in the base housing. The rear member further comprises locating means for mounting at least one fan to the rear member, and air exhausting means for direction an air flow through the rear member.

Preferably, the sterilizer support base also includes at least one fan unit mounted via the locating means to the rear member, and a switch mounted to the base housing for operating the at least one fan unit to cause an air flow through the rear member via the air exhausting means. The switch may be mounted so that it is activated by the holding shelf when the holding shelf is retracted to a position within the cavity.

The sterilizer support base also preferably includes a heating element mounted within the cavity for directing heat toward a holding surface of the holding shelf. The heating element may comprise an electrical strip heater and/or a heating tube for directing a heated fluid through the cavity. The electrical strip heater is preferably operated by the switch.

In one embodiment of the present invention, the sterilizer support base includes at least one securing panel upwardly attached at an edge of the top member to define a chassis of the sterilizer unit for holding components of the autoclave together in an assembly. Each of the sterilizer support base and the holding shelf may be formed as a single piece of sheet steel, or alterntively, as a plastic molding.

### BRIEF DESCRIPTION OF THE DRAWINGS

A more complete understanding of the invention may be obtained by reading the following description of specific illustrative embodiments of the invention in conjunction with the appended drawing in which:

FIG. 1 provides a perspective view of an autoclave support base, with the holding shelf extended and as seen from a right front side;

FIG. 2 provides a perspective view of the support base of FIG. 1 as seen from a right rear side, and illustrating a holding element positioned in the holding shelf;

FIG. 3 provides a perspective view of the support base of FIG. 1 as seen from the right front side, further depicting placement of an autoclave on the top surface of the support base;

FIG. 4 provides a front view of the support base of FIG. 1 further depicting placement of an autoclave on the top surface;

FIG. 5 illustrates an element of the present invention including a spring catch mechanism for retaining the holding shelf of the support base in a retracted position;

FIGS. 6(a), 6(b) present an alternative shelf and holding element;

FIG. 7 shows an alternative embodiment of the support base having an integral autoclave chassis defined by one or more base housing securing panels;

FIG. 8 presents a perspective view from a top elevation of an actively ventilate autoclave support base in accordance with the present invention;

FIG. 9 presents a top and side views of the inventive autoclave support base of FIG. 8; and

FIG. 10 presents a perspective view from a bottom elevation to illustrate a heating element of the inventive autoclave support base.

In the various figures, like reference numerals wherever possible designate like or similar elements of the invention.

### DETAILED DESCRIPTION OF THE PREFERRED EMBODIMENTS

The following detailed description includes a description of the best mode or modes of the invention presently contemplated. Such description is not intended to be understood in a limiting sense, but to be an example of the invention presented solely for illustration thereof, and by reference to which in connection with the following description and the accompanying drawing one skilled in the art may be advised of the advantages and construction of the invention. For example, while the description is made with reference to autoclave sterilizers, application to other types of sterilizers, such as dry-heat and chemical vapor sterilizers, is fully contemplated within the scope of the invention.

The invention is described in terms of embodiments foreseen by the inventor for which an enabling description was available, notwithstanding that insubstantial modifications of the invention, not presently foreseen, may nonetheless represent equivalents thereto.

Autoclave Base

A right front perspective view of an inventive autoclave support base 100 is shown in FIG. 1. Support base 100 includes a base housing 110 and a holding shelf 130, the holding shelf 130 being slidably mounted in a cavity defined by a top surface 111, a left support member 112 and a right support member 113 of the base housing 110. Holding shelf 130 further includes a gripping surface 138a, which flows downwardly from a front edge 138 of holding shelf 130. Gripping surface 138a provides a convenient handle for extending and retracting holding shelf 130.

Base housing 110 and holding shelf 130 may each be formed in a conventional manner from a single piece of sheet steel. Alternatively, base housing 110 and holding shelf 130 may each be formed as conventional plastic moldings.

Although left and right support members 112, 113 are shown in FIG. 1 as extending continuously along left and right edges of top surface 11, one skilled in the art will readily recognize that support members may be configured in a variety of alternative ways to support top surface 111. For example, support members 112, 113 may be alternatively configured as legs positioned at left and right or front and back edges of top surface 111. The legs may be positioned inwardly from rather than directly at the left and right or front and back edges.

FIG. 2 shows a right rear perspective view of the support base 100 of FIG. 1. As illustrated in FIG. 2, base housing 110 further includes a rear support member 114 which is attached to a rear edge 124 of the base housing 110. Rear support member 114 is similar in shape and size to legs 112a, 113a earlier described. When placed upon a supporting surface (not shown), rear support member 114 provides additional rigidity to the top surface 111 of base housing 110. Optionally, rear support member 114 may further include a base flange 115, which together with support member flanges 116 provides an increased contact area between the base housing 110 and the supporting surface. Optionally, base flange 115 and support member flanges 116 may each contain one or more apertures 125 for fixedly attaching base housing 110 to the supporting surface using conventional fasteners.

Holding shelf 130 of FIG. 2 includes a holding surface 132 (partially obscured) which is depressedly positioned with respect to holding shelf edges 138. Surfaces 131 defined between edges 138 and holding surface 132 are approximately perpendicular to holding surface 132 and serve to confine items placed on holding surface 132 in proximity to one or more edges 138.

As shown in FIG. 2, holding shelf 130 may also optionally include a holding element 139, which is positioned near edges 138 and supported by holding surface 132. Holding element 139 may be fixedly fastened to holding surface 132 by any of a variety of fastening techniques, or alternatively, may be integrally formed as part of holding shelf 130.

In FIG. 2, holding element 139 has a drying surface 150 which is further defined by a plurality of lateral troughs 133 and lateral crests 134. Items that have been sterilized in the autoclave, for example, may be positioned to rest across lateral crests 134 in order to promote air flow through lateral troughs 133 for improved drying and cooling of the sterilized items. Alternatively, lateral troughs 133 may be used to organize and hold some of the drying items (for example, such as dental picks and other slender instruments). Condensate that forms on the drying items may effectively drain into lateral troughs 133 to facilitate drying.

In order to more effectively remove condensate from the holding element 139, lateral troughs 133 may be pitched such that condensate drains to holding surface 132. Holding surface 132 may also be pitched so that this condensate may be led to a perforation in holding surface 132 for draining.

FIG. 3 illustrates an embodiment of the present invention in which top surface 111 of the support base 100 supports an autoclave 200. Autoclave 200 is, by way of example, a front-loading unit having a sterilizing chamber 220 and a sealing door 210. Holding shelf 130 of the support base 100 is conveniently positioned for loading items in and unloading items out of sterilizing chamber 220.

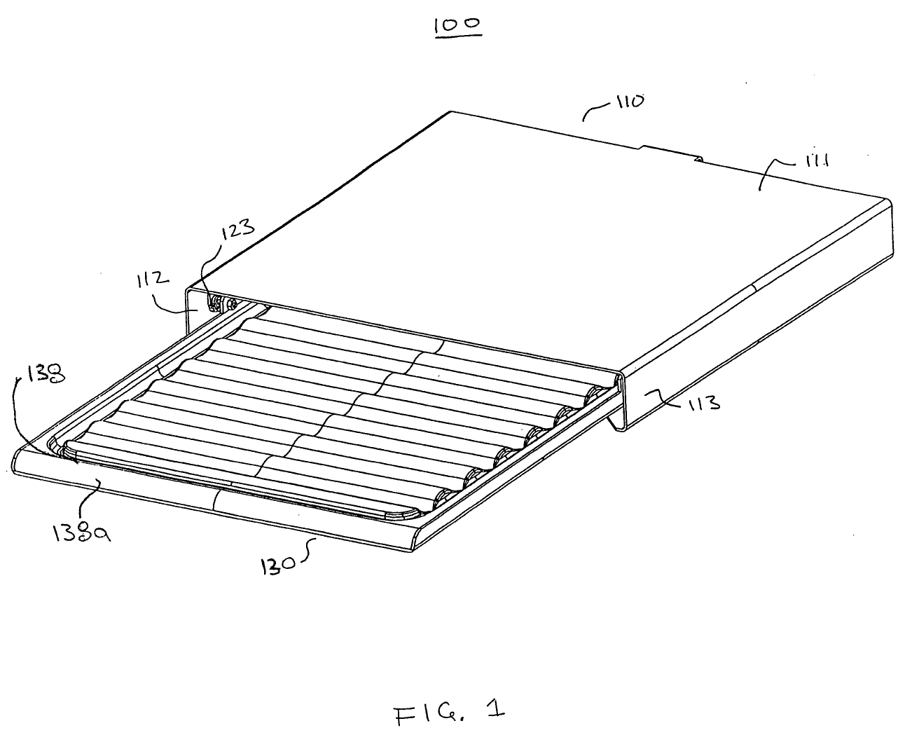

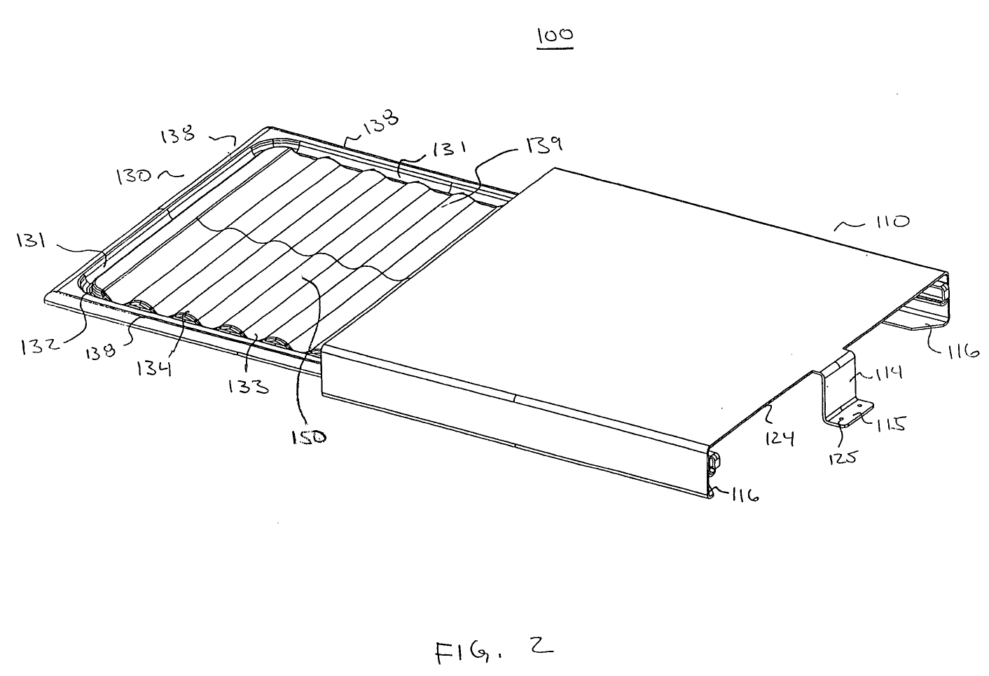

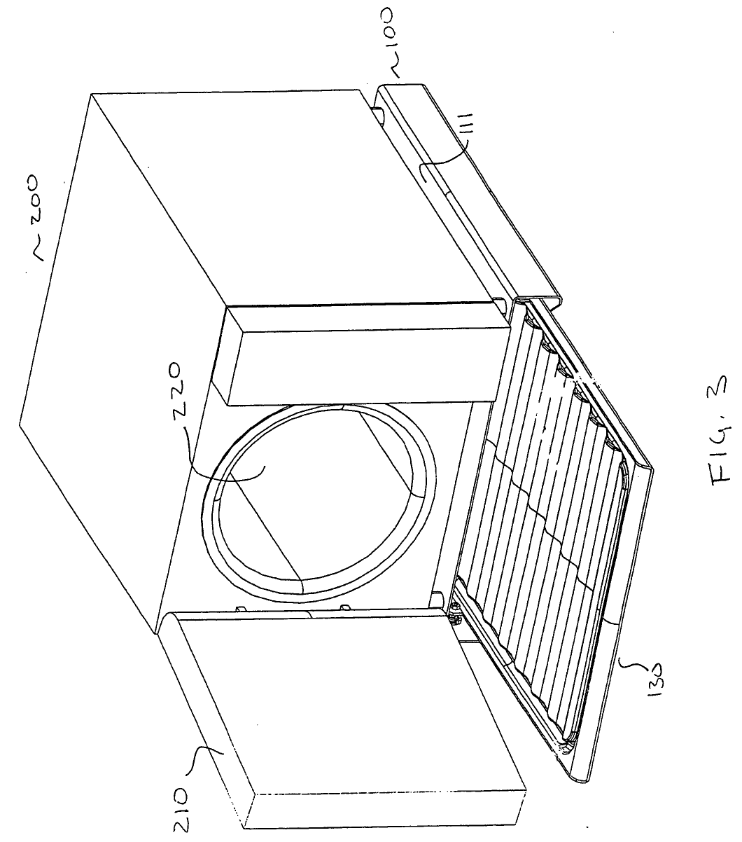

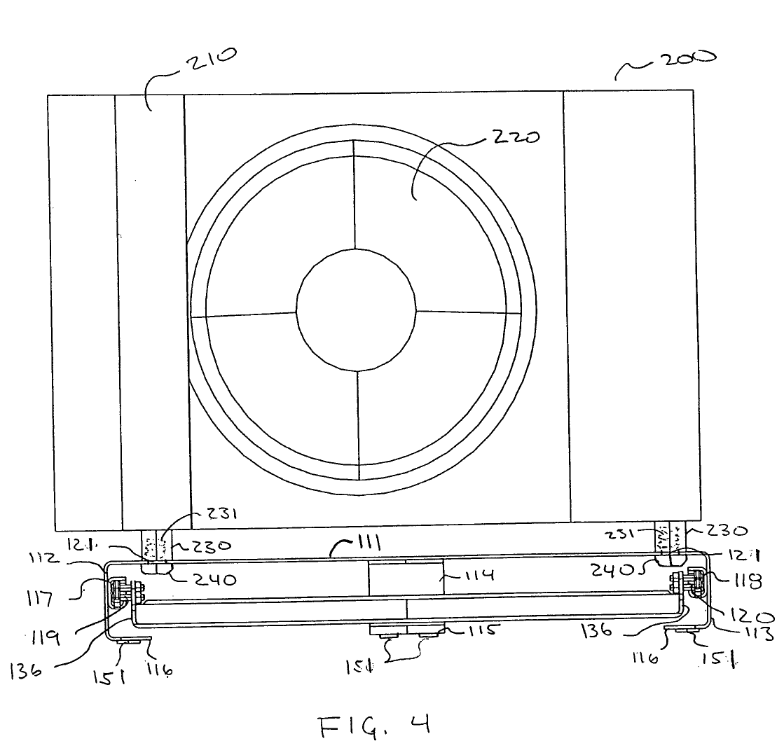

FIG. 4 provides a front view of the embodiment of FIG. 3. FIG. 4 illustrates conventional pull-out slides (sometimes referred to as drawer slides) that include a left track 117 fastened to left support member 112 and a right track 118 fastened to right support member 113. Glide wheel assemblies 119 and 120 are respectively retained by the tracks 117 and 118, which are fixedly attached to side surfaces 136 of holding shelf 130. Glide wheel assemblies 119 and 120 move within tracks 117 and 118 in order for holding shelf 130 to move from a retracted position mostly within the base housing 110 to an extended position mostly outside base housing 110. The extended position of holding shelf 130 is illustrated in FIGS. 1-3. As illustrated in FIG. 1, for example, retention stops 123 may be fitted at front ends of tracks 117 and 118 in order to prevent holding shelf 130 from being over-extended from base housing 110. Retention stops 123 (not shown) may also be optionally fitted at the rear ends of tracks 117 and 118 in order to more effectively position holding shelf 130 in the retracted position. Such track and glide assemblies are well-known in the art, and are readily available (for example, as part number C2132-20DOP from Accuride International Inc. of Sante Fe Springs, Calif.).

FIG. 4 also illustrates how autoclave 200 may be secured to base housing 110. Autoclave 200 includes a plurality of feet 230, each of which contains a threaded insert 231. Top surface 111 of base housing 110 included a number of apertures 121 whose centerlines are positioned to align with centerlines of the threaded inserts 231 when autoclave 200 is appropriately positioned on top surface 111. Once positioned on top surface 111, autoclave 200 may be secured by inserting bolts 240 through the apertures 121 to mate with threaded inserts 231, and then appropriately tightening the bolts 240 until autoclave 200 is secured to top surface 111. Autoclave 200 may be secured to base housing 110 by a variety of other equivalent means. For example, tabs may be punched in top surface 111 to mate with slots in a front pair of autoclave feet 230, so that only a rear pair of autoclave feet 230 are fastened by inserting bolts 240 through a matching pair of rear apertures 121. Alternatively, lateral movement of autoclave 200 may be limited by providing indented or embossed regions in top surface 111 for restraining feet 230 of autoclave 200.

As earlier noted, base flange 115 and support member flanges 116 may each contain one or more apertures 125 for fixedly attaching base housing 110 to a supporting surface using conventional fasteners. Alternatively, and as illustrated in FIG. 4, base flange 115 and support member flanges 116 may be fitted with friction pads 151 to reduce lateral movement between flanges 115, 116 and the supporting surface. Friction pads 151 may be adhesively or mechanically fastened to flanges 115, 116, and may be fabricated from a variety of suitable materials including rubber and cork. As an additional alternative, flanges 115, 116 may optionally be fitted with vertically-adjustable feet incorporating pads 151. Such adjustable feet are well-known in the art, and provide an advantage in stabilizing the autoclave unit on an uneven work surface.

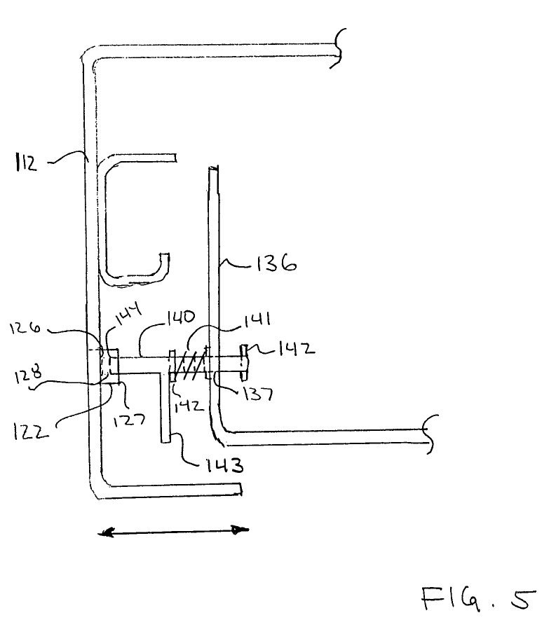

FIG. 5 presents an enlarged cross-sectional view taken near the front edge of support base 100 illustrated in FIG. 4. As shown in FIG. 5, a spring catch 140 is inserted through aperture 137 to be retained in side surface 136 of holding shelf 130 by retaining washers 142. Retaining washers 142 are affixed to spring catch 140 by any of a variety of conventional means.

When holding shelf 130 is retracted within base housing 110, force applied by spring 141 causes spring catch 140 to extend such that a distal end 144 of spring catch 140 is secured behind retaining tab 122 in left support member 112. In this position, holding shelf 130 is secured to remain in a retracted position within base housing 110. When it is desired to extract holding shelf 130, disengaging arm 143 may be manipulated to compress spring 141, moving spring catch 140 to a position where distal end 144 is no longer captive behind retaining tab 122. In this catch position, holding shelf 130 may be easily extracted from base housing 130.

It should be noted that retaining tab 122 may be, for example, punched from left support member 112 such that a front edge 126 of retaining tab 122 is coplanar with an inner surface of left support member 112, where front edge 126 is positioned outward from retaining edge 127. In this configuration, a cam surface 128 defined by edges 126, 127 acts to compress spring 141 and move catching spring 140 toward side surface 136 when force is applied to return holding shelf 130 to its retracted position. By such means, distal end 144 may be automatically moved behind retaining tab 122 without manually operating disengaging arm 143.

Various other mechanisms may be employed for retaining holding shelf 130 within base housing 110. For example, detents (not shown) may be incorporated in a lower portion of track 117, such that glide wheels from glide wheel assemblies 119 may be moved vertically against gravity force and horizontally rearward to rest behind and be retained by the detents. Retained glide wheels may then be released by moving the wheels vertically against gravity force and horizontally forward from the detents. Such gravity catches are well-known in the art, and are available, for example, in drawer slide part number D2132-20D from Accuride International Inc. Alternatively, conventional magnetic catches may be employed to secure holding shelf 130 in a retracted position within base housing 110. Catches may also be positioned to retain holding shelf 130 at one or more intermediate positions of extension from base housing 130.

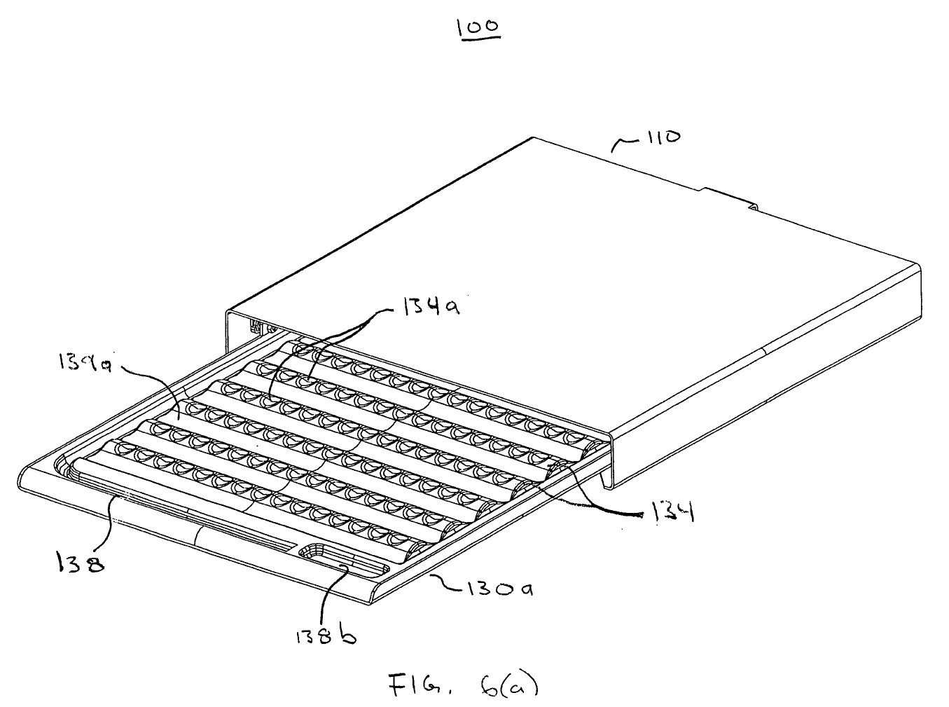

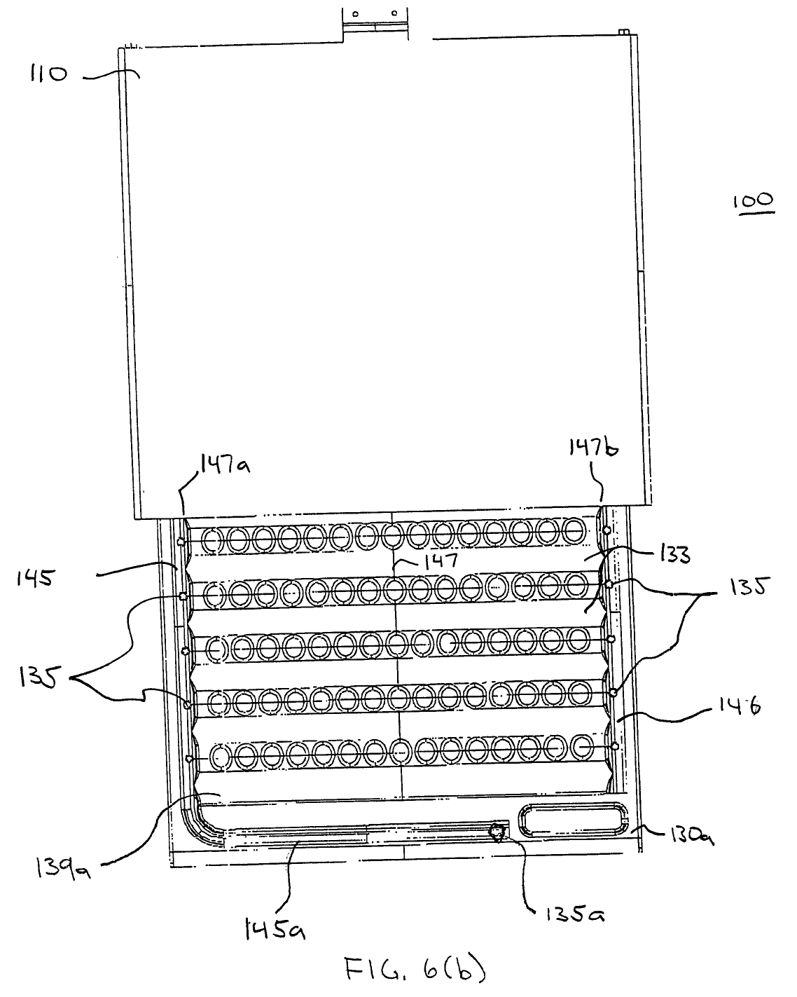

FIGS. 6(a), 6(b) illustrate an alternative holding element 139a and holding shelf 130a for support base 100. Holding element 139a of FIG. 6(a) is positioned in holding shelf 130a, which extends from base housing 110. Lateral crests 134 of holding element 139a are dimpled to include regularly-spaced indentations 134a. Indentations 134a provide a means for positioning items on crests 134 for drying. This means will be particularly useful, for example, in positioning thin, axially-oriented items such as dental instruments on the lateral crests.

As further illustrated in FIG. 6(a), holding shelf 130a includes cavity 138b near a right side of front edge 138. Cavity 138b may be used to hold small items not easily positioned on crests 134 for drying. Alternatively, cavity 138b may be used as an additional grip for extending and retracting holding shelf 130a.

Referring to FIGS. 2, 6(a) and 6(b), holding elements 139, 139a may be fashioned from a variety of materials, including sheet steel and molded plastic, and may be either removably or fixedly mounted within holding shelves 130, 130a, respectively. Holding elements 139, 139a may also optionally be integrally formed with holding shelves 130, 130a.

FIG. 6(b) presents a top view of the holding element 139a positioned in holding shelf 130a of base housing 110. Perforations 135 are located along a bottom portion of channels 145, 146, respectively located at left and right edges 147a, 147b of holding element 139a. Channels 145, 146 are positioned to receive condensate drainage from holding element 139a, which may be facilitated, for example, by pitching lateral troughs from centerline 147 downward towards channels 145, 146. Perforations 135 provide a means for draining condensate received by channels 145, 146.

Alternatively, in lieu of perforations 135, channel 145 may be pitched downwards toward perforation 135(a), which is positioned in channel extension 145a near a front edge of holding shelf 130a. This alternative configuration provides the advantage of positioning condensate drainage from holding shelf 130a near the front edge of the shelf where it can be more easily and confinedly dispersed. In this configuration, for example, channel 146 may be omitted, and lateral troughs 133 may be pitched downward from right edge 147b of holding element 139a toward channel 145.

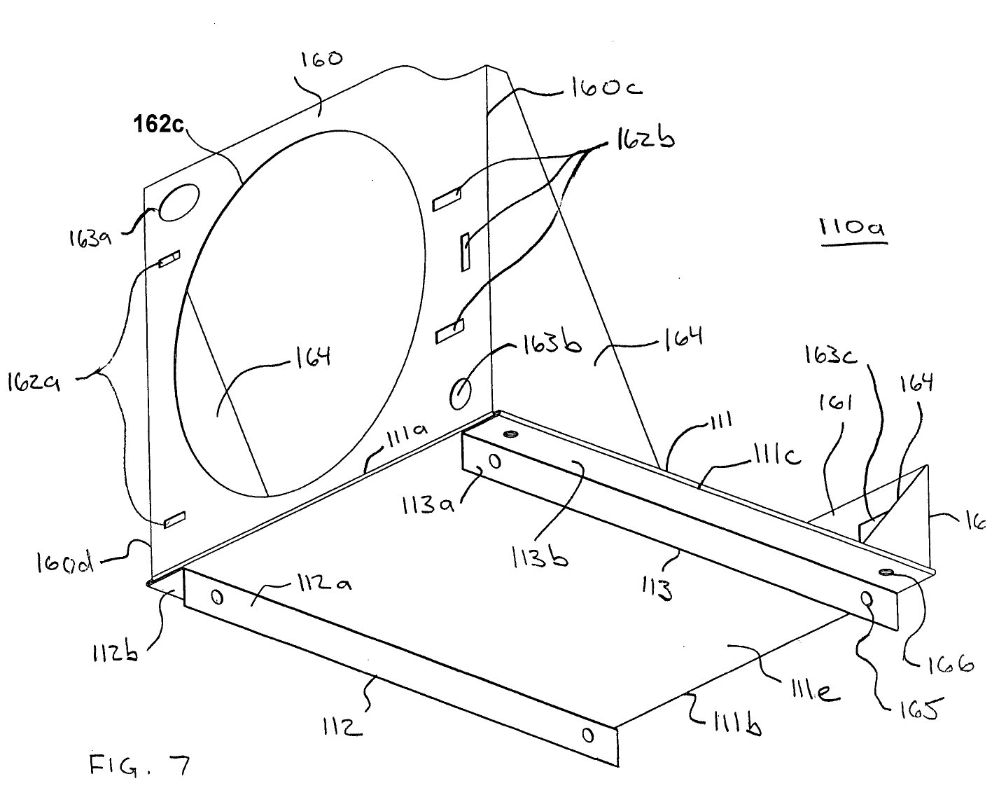

FIG. 7 presents a perspective view of a second base housing embodiment 110a. In base housing 110a, securing panels 160, 161 extend upwardly from top and rear edges 111a, 111b of top surface 111, respectively. Securing panels 160, 161 effectively define an internal chassis for autoclave 200. In other words, the internal and external components of autoclave 200 may be assembled to securing panels 160, 161 so that base housing 110a becomes an integral part of autoclave 200.

A illustrated in FIG. 7, front securing panel 160 includes autoclave chamber access aperture 162c to permit access to the autoclave chamber 220 of FIG. 2. In addition, hinge mount apertures 162a and latch mount apertures 162b respectively provide mounting points for hinge and latch hardware associated with autoclave 200. Access apertures 163a, 163b provide access for controls on the front panel of autoclave unit 200. Access aperture 163c may be provided on rear securing panel 161 for a similar purpose.

Gusset plates 164 are optionally attached at right and left edges 160c, 160d of front securing panel 160 and at right and left edges 111c, 111d of top surface 111 to make securing panel 160 more rigid. Gusset plates 164 may also be optionally attached at right and left edges 161c, 161d of rear securing panel 161 and right and left edges 111c, 111d of top surface 111. Although base housing 110a of FIG. 7 is illustrated with front securing panel 160 and rear securing panel 161, embodiments including only one of the front and rear panels 160, 161, as well as embodiments including one or more side panels attached at side edges 111c, 111d, are fully contemplated and within the scope of the present invention.

It should also be noted that, securing panels 160, 161 may be alternatively configured as an external chassis for confining autoclave 200 to top surface 111 of FIG. 7. In this alternative configuration, securing panels 160, 161 may be fastened to components affixed to the exterior of autoclave 200 (for example, fastening external hinge and latch hardware respectively through apertures 162a, 162b of securing panel 160.) In addition, gusset plates 164 and securing panels 160, 161 may be positioned in close proximity to exterior surfaces of autoclave 200 in order to restrict lateral movement of autoclave 200 on top surface 111. Other means (for example, as described in conjunction with FIG. 4) may be additionally employed to retain autoclave 200 against top surface 111.

FIG. 7 also illustrates an alternate embodiment for support members 112,113 of FIG. 1. In FIG. 7, support members 112a, 112b and support members 113a, 113b each form an L-shaped member 112, 113 for supporting top surface 111. Members 112b, 113b are respectively attached at edges 111d, 111c and to bottom surface 111e so that members 112a, 113a are vertically positioned with respect to top surface 111, and inwardly positioned with respect to edges 111d, 111c. Members 112a, 113a are also parallel to edges 111c, 111d, and to each other.

Apertures 165 in members 112a, 113a may be used to fasten tracks 117, 118 (not shown) on interior surfaces of members 112a, 113a. Alternatively, tracks 117,118 may be fastened to members 112a, 113a by a variety of other means including spot welding and gluing. Apertures 166 may be used for securing members 112, 113 to bottom surface 111e. Again, alternative fastening means such as spot welding and gluing may be used. Although members 112, 113 may simply be fastened to edges 111d, 111c, for example, as a folded construction from a single piece of sheet metal, it may be advantageous to provide additional fastening between members 112b, 113b and bottom surface 111e for added strength. If such additional fastening means are employed, apertures 166 may be alternatively used, for example, to mount adjustable feet for positioning base housing 110a on an uneven work surface. As depicted, base housing 110a may be fashioned from one or more pieces of folded sheet steel using conventional assembly methods. For strength and corrosion resistance, stainless steel is a preferred material for base housings 110, 110a, although molded plastics and other materials may be employed as well.

Ventilated Drawer

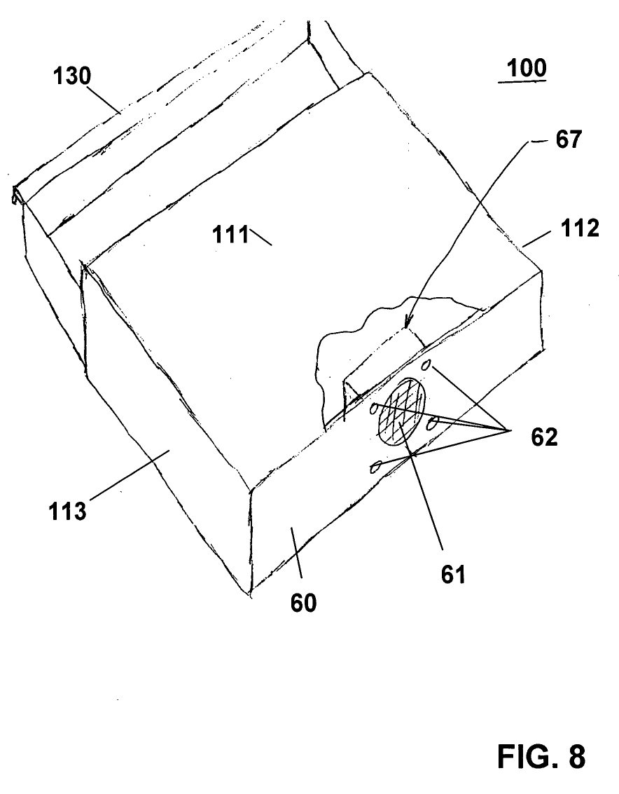

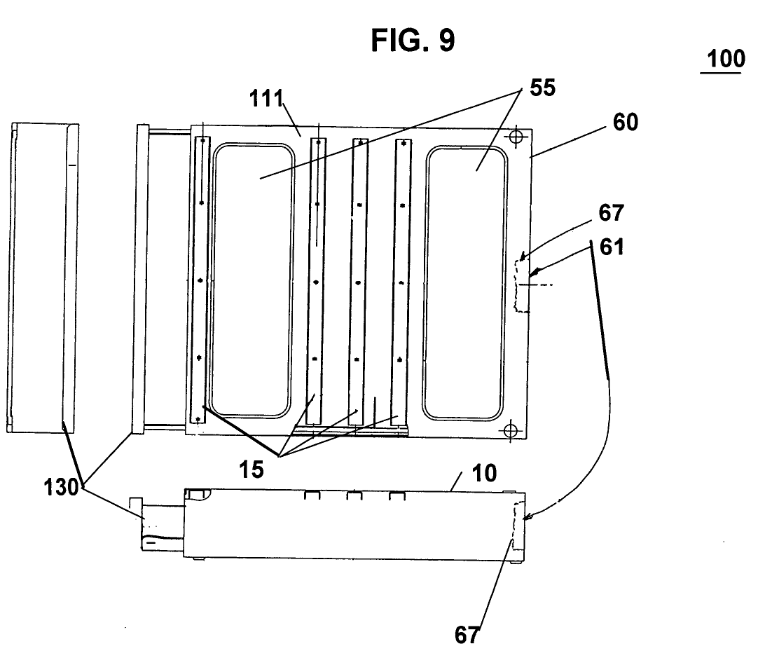

As illustrated by FIGS. 8 and 9, autoclave support base 100 comprises top surface 111, left and right support members 112, 113, and holding shelf 130. In addition, rear member 60 is coupled to top surface 111. Top surface 111 and members 112, 113 and 60 create an enclosure for slidably receiving holding shelf 130. As illustrated for example in FIG. 9, top surface 111 may include reinforcing members 15 for rigidly supporting the autoclave unit, and may include embossed regions 55 for additional rigidity and for locating the feet of an autoclave unit positioned on top surface 111.

In rear member 60, apertures 61 and 62 are provided for mounting a standard muffin fan 67 (such as is available, for example, from Northern Tool and Equipment Co. of Burnsville, Minn.), preferably in the interior of the enclosure defined by top surface 111 and members 111,112 and 60. Stops (not shown) are included, for example, along interior surfaces of support members 111,112 so that holding shelf 130 when retracted in the enclosure will not interfere with the muffin fan 67. A microswitch (not shown) is also positioned along one interior surface of one of support members 111,112 so that the muffin fan 67 will be turned on when the holding shelf 40 is retracted. Air flow will normally be directed out of the enclosure through aperture 61.

In order to promote ventilation, holding shelf 130 will normally have a smaller vertical profile than support members 111,112. For example, holding shelf 130 may have a vertical height of about 1¼ inches, while support members 11, 112 may have vertical heights of about 4 inches.

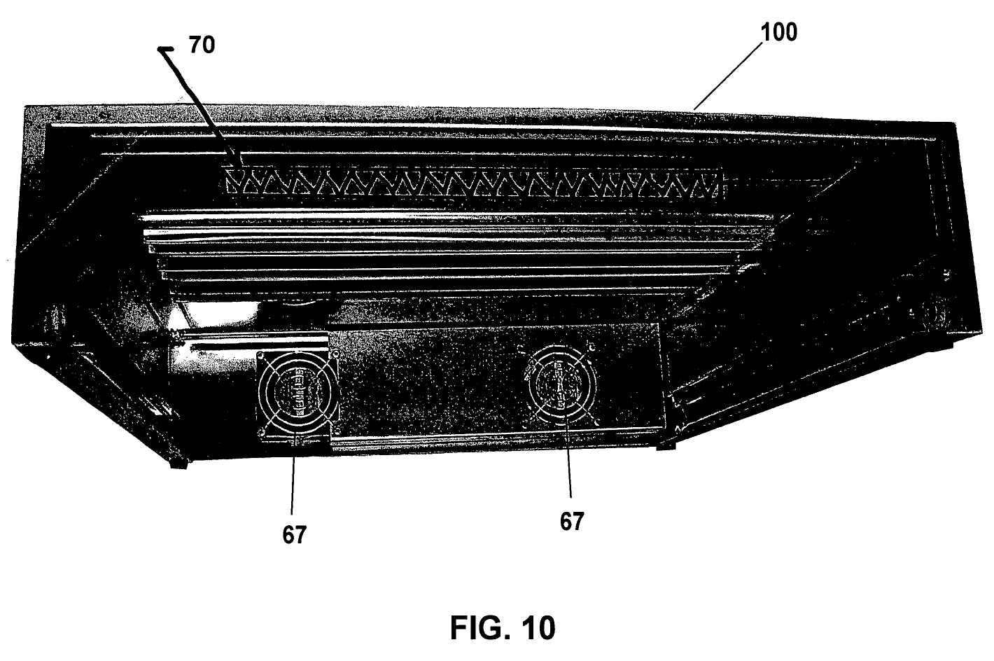

It is envisioned that more than one muffin fan 67 may be mounted along the surface of rear member 60 (see, e.g. FIG. 10).

In one embodiment of the present invention, apertures 61 and 62 are not fully formed in rear member 60, but rather ½ shear cuts are made in rear member 60 that effectively define circumferences of apertures 61 and 62. In this embodiment, apertures 61 and 62 can be created by punching the interior portions within these circumferences out from rear member 60. This feature allows the support base to be produced without the ventilation feature initially for easy retrofitting of muffin fans 67 at a future time. A similar feature may be employed for mounting the microswitch.

The support base 100 disclosed by U.S. Provisional Patent Application S/N 60/493,843 may be further improved by incorporating a resistive heating element 70 to further increase the rate of drying for sterilized items placed on the holding surface of the holding shelf 130. Suitable electrical strip heaters may be incorporated as resistive heating element 70, for example, such as are available from CHROMOLOX of Pittsburgh, Pa. Heating element 70 may be switched together with muffin fans 67 by the microswitch, or may be switched by alternate switch means provided in support base 100.

It is further envisioned that the ventilating and heating features of the support base 100 may be may be incorporated in a support base having an integral autoclave chassis as illustrated for example in FIG. 7. In this case, the autoclave unit is assembled on the chassis such that the slidable holding shelf 130 is an integral part of the autoclave unit. As the autoclave provides additional heating sources (for example, generated steam), it is further contemplated that heating element 70 of FIG. 3 may alternatively comprise a heating tube for directing steam or some other heated fluid for increasing increase the rate of drying for sterilized items placed on the holding surface of the holding shelf 130.

1. A sterilizer support base, the support base comprising:
a base housing, said base housing having:
a top member for receiving and supporting a sterilizer unit,
left and right support members respectively and downwardly attached from the top member, and
a rear member downwardly attached from the top member and having locating means arranged for mounting at least one fan unit to the rear member,
wherein the top member and the left and right support members define a cavity in said base housing; and
a holding shelf, said holding shelf being slidably mounted within the cavity in said base housing;
wherein the rear member further comprises air exhausting means arranged for directing an air flow through the rear member.

2. The sterilizer support base of claim 1, wherein the locating means comprises at least a first aperture, and the air exhausting means comprises at least a second aperture.

3. The sterilizer support base of claim 1, wherein the locating means comprises ½ shear cuts in the rear member defining at least a first aperture, and the air exhausting means comprises ½ shear cuts in the rear member defining at least a second aperture.

4. The sterilizer support base of claim 2, further comprising:
at least one fan unit mounted to the rear member via the locating means, wherein the at least one fan unit may be operated to cause the air flow directed through the rear member via the air exhausting means.

5. The sterilizer support base of claim 1, further comprising:
second locating means arranged for mounting a switch to the base housing for operating at least one fan unit.

6. The sterilizer support base of claim 5, wherein one of the left and right support members includes the second locating means.

7. The sterilizer support base of claim 5, wherein the second locating means comprises at least one a third aperture.

8. The sterilizer support base of claim 5, wherein the second locating means comprises ½ shear cuts defining at least a third aperture.

9. The sterilizer support base of claim 4, further comprising:
a switch mounted to the base housing via a second locating means of the base housing, wherein the switch controls operation of the at least one fan unit.

10. The sterilizer support base of claim 9, wherein the switch is mounted to be engaged by the holding shelf when the holding shelf is retracted to a position within the cavity.

11. The sterilizer support base of claim 1, further comprising:
a heating element mounted within the cavity for directing heat toward a holding surface of the holding shelf.

12. The sterilizer support base of claim 11, wherein the heating element is mounted on a bottom surface of the top member of the base housing.

13. The sterilizer support base of claim 11, wherein the heating element is an electrical strip heater.

14. The sterilizer support base of claim 4, further comprising:
a switch mounted to the base housing via a second locating means of the base housing, wherein the switch controls operation of the at least one fan unit; and
a heating element mounted within the cavity for directing heat toward a holding surface of the holding shelf.

15. The sterilizer support base of claim 14, wherein the heating element is an electrical strip heater and the switch further controls operation of the heating element.

16. A sterilizer unit comprising:
a sterilizer support base, the support base including:
a base housing, said base housing having:
a top member,
left and right support members respectively and downwardly attached from the top member,
a rear member downwardly attached from the top member and having locating means arranged for mounting at least one fan unit to the rear member, and
at least one securing panel, the at least one securing panel being upwardly attached at an edge of the top member,
wherein the top member and the left and right support members define a cavity in said base housing and the at least one securing panel defines a chassis of the sterilizer unit; and
a holding shelf, said holding shelf being slidably mounted within the cavity in said base housing;
wherein the rear member further comprises air exhausting means arranged for directing an air flow through the rear member.

17. The sterilizer unit of claim 16, wherein the locating means comprises at least a first aperture, and the air exhausting means comprises at least a second aperture.

18. The sterilizer unit of claim 17, further comprising:
at least one fan unit mounted to the rear member via the locating means, wherein the at least one fan unit may be operated to cause the air flow directed through the rear member via the air exhausting means.

19. The sterilizer unit of claim 18, further comprising:
a switch mounted to the base housing via a second locating means of the base housing, wherein the switch controls operation of the at least one fan unit; and
a heating element mounted within the cavity for directing heat toward a holding surface of the holding shelf.

20. The sterilizer unit of claim 19, wherein the heating element is an electrical strip heater and the switch further controls operation of the heating element.

21. The sterilizer unit of claim 19, wherein the heating element is a heating tube for directing a heated fluid through the cavity.

22. The sterilizer support base of claim 1, wherein at least one of said base housing and said holding shelf is formed from a single piece of sheet steel.

23. The sterilizer support base of claim 1, wherein at least one of said base housing and said holding shelf is formed as a plastic molding.

24. The sterilizer support base of claim 1, wherein the holding shelf includes a holding surface depressedly positioned with respect to one or more edges of the holding shelf such that surfaces defined between the one or more edges of said holding shelf and the holding surface may be used to confine items placed on the holding surface.

25. The sterilizer support base of claim 24, further comprising:
a holding element positioned near said one or more edges of said holding shelf and supported by said holding surface.
